(19) United States
(12) Patent Application Publication
(10) Pub. No.: US 2004/0267119 A1
(43) Pub. Date: Dec. 30, 2004

(54) Method for matching transmit voltages of different ultrasonic imaging modes

(76) Inventor: Darwin P. Adams, Lexington, MA (US)

Correspondence Address: PHILIPS INTELLECTUAL PROPERTY & STANDARDS, P.O. BOX 3001, BRIARCLIFF MANOR, NY, 10510, US

(21) Appl. No.: 10/878,178

(22) Filed: Jun. 28, 2004

Related U.S. Application Data
(60) Provisional application No. 60/482,656, filed on Jun. 26, 2003.

(51) Int. Cl. A61B 8/00

(52) U.S. Cl. 600/437

(57) ABSTRACT

Method for matching transmit voltages in imaging modes of a medical ultrasound imaging system having different transmit power requirements in which a common power supply voltage is directed from a power supply to transmitting elements and when switching between the imaging modes on a line-by-line basis, if the transmit power requirement for an imaging mode is less than that provided by the power supply voltage, the transmit waveform for that imaging mode is pulse width modulated, e.g., by directing pulse width modulation (PWM) signals to the transmitting elements. The power supply voltage is not changed between different imaging modes when imaging on a line-by-line basis. The PWM signals are generated by a timing generator based on the transmit power requirements of the imaging mode so that the total power in the waveform generated by the transmitting elements during that imaging mode does not exceed the transmit power thereof.

### CROSS REFERENCE TO RELATED CASES

Applicant claims the benefit of Provisional Application Ser. No. 60/482,656, filed Jun. 26, 2003.

### FIELD OF THE INVENTION

The present invention relates generally to methods for controlling the formation of ultrasonic waves by a medical ultrasound imaging system and more particularly to methods for matching transmit voltages of different imaging modes of a medical ultrasound imaging system.

### BACKGROUND OF THE INVENTION

In medical ultrasound imaging systems, there are often several different imaging modes, such as B-mode, Color and spectral Doppler, which can be operational simultaneously on a line-by-line basis. That is, one line of B-mode can immediately follow a line of Color mode. Each of these modes must be operated at a specific transmit power level dictated by both requirements for patient safety and imaging performance. Transmit power is normally affected by a combination of transmit amplitude, transmit waveform shape, the duration of the transmit burst, and pulse repetition rate of transmit bursts. Since the burst repetition rate and duration of the transmit burst are often defined by the operational mode for performance reasons, it is not often practical to vary these parameters in order to control transmit power. Thus, typically the transmit amplitude and transmit waveform shape are varied.

Many factors including performance, complexity and cost affect the design of ultrasound transmit circuitry. One common design is a simple saturating switch (FET) which alternately connects the load to ground or to power supply rail(s). In such a design, transmit power is normally controlled by adjusting the voltage at the power supply rail(s) to affect the amplitude of the transmit waveform. A second common design, which is more sophisticated but also more complex and expensive, is a linear amplifier (ARB). In this design, the linear amplifier is provided a fixed power supply rail voltage and the transmit waveform amplitude is controlled by adjusting the gain of the linear amplifier or the level of the control waveform driving the linear amplifier. The linear amplifier design provides excellent control not only of transmit power, but of other performance aspects of the transmit waveform. However, its cost and complexity have generally prevented its use in lower cost ultrasound products.

The present invention relates to the control of transmit power for the saturating switch or FET type transmit circuitry and provides a method for controlling transmit power by pulse width modulation rather than by adjustments to power supply rails connected to the FET transmit drivers. While supply rail control is effective, it generally is very difficult, if not impossible, to make rapid and reliable changes in supply voltage. When an ultrasound system is operating in a multi-mode state such as B-mode/Color, Duplex or Triplex, the transmit power must be altered on a line-by-line basis with consistent and accurate control of transmit power for each line according to its mode. Since changes in supply voltage cannot generally occur on a line-by-line basis, it is a common compromise to provide a single supply voltage level which is low enough to satisfy patient safety for the worst case mode, i.e., the mode with the lowest transmit voltage. In this situation, it is a disadvantage that all modes use the same supply voltage.

Ultrasound systems which operate in this manner can often suffer performance issues when imaging in multiple modes such as B-Mode/Color. For example, when operating in B-mode/Color, both a B-mode and a Color image are formed. Since Color normally uses a longer duration transmit burst than B-mode, it must operate at a lower transmit amplitude than B-mode. In this case, the transmit voltage for B-mode would be set below its optimum value since the level must be determined, for patient safety reasons, to that of the Color mode. A drawback of reducing the transmit voltage of the B-mode below its optimum value is that the B-mode sensitivity drops when Color is activated. This problem becomes even more severe when operating in Duplex or Triplex modes since Doppler burst duration tends to be long and transmit amplitude for this mode tends to be very small.

### SUMMARY OF THE INVENTION

It is an object of the present invention to provide new methods for matching transmit voltages in different imaging modes of a medical ultrasound imaging system on a line-by-line basis.

It is an object of the present invention to provide new and improved methods for controlling transmit power in different imaging modes for medical ultrasound systems using saturating switch type transmit circuit designs operating from fixed or slowly variable power supply voltages.

It is another object of the present invention to provide new and improved methods for matching transmit voltages in different imaging modes of a transducer of an ultrasound system in which each mode can operate at an optimum transmit power.

It is still another object of the present invention to improve the operation of an ultrasonic transducer at different modes requiring different transmit power by pulse width modulating the transmit waveforms and thereby avoiding the need to change the power supply voltage.

It is yet another object of the present invention to control an ultrasonic transducer having pulse width apodization circuitry to use the same circuitry for pulse width modulation to provide an inexpensive method for matching transmit voltages of different imaging modes.

It is another object of the present invention to provide a new medical ultrasound imaging system operable in multiple imaging modes having different transmit power requirements.

In order to achieve these objects and others, a method for matching transmit voltages of different imaging modes of an ultrasonic transmitter having different transmit power requirements in accordance with the invention entails directing the same power supply voltage to all of the transmitting elements, operatively switching between the imaging modes on a line-by-line basis and only when the requirement for the transmit power for an imaging mode is less than that provided by the power supply voltage, pulse width modulating the transmit waveform for that imaging mode, for example, by directing pulse width modulation signals to all of the transmitting elements.

By pulse width modulating the transmit waveform generated by the transmitting elements during those imaging modes that should not receive the full power output, the same power supply can be directed to all of the transmitting elements regardless of the imaging mode of the ultrasound imaging system so that changing the power supply voltage for different imaging modes is not required. The drawbacks associated with operatively changing the power supply voltage for different imaging modes are therefore avoided.

Moreover, each imaging mode can operate at its optimum power without requiring any reduction in the voltage to the transmit circuitry which might result from operation of the system for a different imaging mode requiring a lower transmit power.

A significant advantage is further achieved if the circuitry for pulse width apodization in the transmitter, which is a known feature of prior art transducers, is used to pulse width modulate the transmit waveform based on the transmit power requirement of each imaging mode.

An ultrasonic transducer applying the method described above includes pulse width modulation circuitry and a system for matching transmit power in different imaging modes of the transmitter which is coupled to the pulse width modulation circuitry. The imaging modes are realized on a line-by-line basis with each line of a frame capable of having being operable in a different imaging mode. The system includes a mechanism for directing the same power supply voltage to all of the transmitting elements and a mechanism for switching between the imaging modes of the transducer. The pulse width modulation circuitry pulse width modulates the transmit waveform for one of the imaging modes and then directs a pulse width modulation signal to all of the transmitting elements only when the transmit power for that imaging mode is less than that provided by the full power supply voltage. This avoids the need to change the power supply voltage between imaging modes having different transmit power requirements.

In a preferred embodiment, the pulse width modulation circuitry is the same as the pulse width apodization circuitry which may be used in the transducer.

### BRIEF DESCRIPTION OF THE DRAWINGS

The invention, together with further objects and advantages hereof, may best be understood by reference to the following description taken in conjunction with the accompanying drawings, wherein like reference numerals identify like elements and wherein:

FIG. 1 is a diagram showing the circuitry for matching transmit voltages in different imaging modes of a medical ultrasound imaging system in accordance with the invention.

FIG. 2A shows an exemplary waveform generated by transmit FETs.

FIG. 2B shows another waveform generated by transmit FETs.

FIG. 2C shows a waveform generated by transmit FETs in accordance with the invention.

FIG. 3 is a flow chart of a method in accordance with the invention for controlling the transmit voltages of a medical ultrasound imaging system operable in multiple modes.

### DETAILED DESCRIPTION OF THE PREFERRED EMBODIMENTS

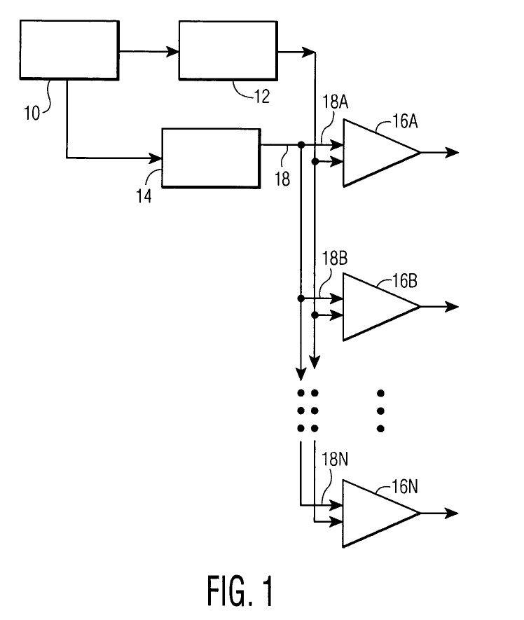

Referring to FIG. 1, the relevant portion of a medical ultrasound imaging system in accordance with the invention which is used to matching transmit voltages of different imaging modes is shown. Matching of the transmit voltages is realized by a control computer or processor 10 which stores events for the control changes and the events for each line, and may also optionally generate such events. The control computer 10 directs the required event commands to a transmit power supply 12 and a timing generator 14. The transmit power supply 12 can provide a full power waveform to all of a plurality of transmit FETs 16A, 16B, . . . 16N via power supply rails. Instead of transmit FETs, other types of transmitting elements may be used in the ultrasound imaging system. The ultrasound imaging system can also include other components for generating the event commands for providing a desired transmit waveform such as disclosed in U.S. Pat. No. 4,937,767, the entire disclosure of which is incorporated by reference herein.

The timing generator 14 generates a timing signal or pulse width modulation signal for the transmit FETs 16A, 16B, . . . 16N and sends the signal over a common connection 18 with a branch 18A, 18B, . . . 18N leading to each transmit FET 16A, 16B, . . . 16N in the ultrasound imaging system. The timing generator 14 determines the number of pulses in each burst, the pulse train frequency, the pulse width and the delay (used for focusing) and generates an appropriate timing signal which will cause the transmit FETs 16A, 16B, . . . 16N to generate the desired waveform from the power supply upon receiving the timing signal.

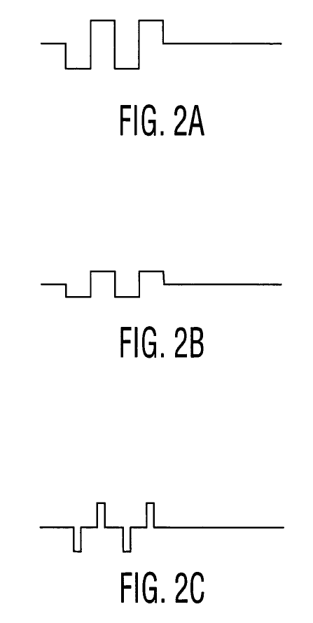

As shown in FIG. 2A, the timing generator 14 can direct a timing signal to cause the transmit FETs 16A, 16B, . . . 16N to generate a full power waveform (with 2 pulses in a burst). This waveform would be generated when the transmit power for an imaging mode is equal to or greater than that provided by the power supply voltage, i.e., the full power supply voltage can be used in that imaging mode.

However, when the transmit power of the imaging mode is less than that provided by the power supply voltage, the full power supply voltage cannot be used in that imaging mode. Therefore, in the prior art, the power supply voltage is reduced to thereby cause the reduction in the power of the waveform as shown in FIG. 2B. A reduction in the power supply voltage being supplied to the transmitting elements has significant drawbacks, namely, that the same reduced power supply voltage must be used for all imaging modes if the imaging system is not capable of switching power supply voltages for the different imaging modes on a line-by-line basis. This reduces the voltage for imaging modes which operate at higher transmit voltages.

To overcome this problem, in accordance with the invention, a timing or pulse width modulation signal is generated by the timing generator 14 to cause the waveform generated by the transmit FETs 16A, 16B, . . . 16N to have a reduced width as shown in FIG. 2C (in comparison to the full power waveform shown in FIG. 2A and the waveform at the reduced power supply voltage as shown in FIG. 2B). This is achieved without reducing the power supply voltage provided over the power supply rails to the transmit FETs 16A, 16B, . . . 16N when switching between imaging modes on a line-by-line basis. As such, instead of reducing the power supply voltage to accommodate the transmit power requirements of all possible imaging modes, the width of the pulse in each burst is reduced. The same power supply voltage is thus used for all imaging modes and is not changed. However, when switching between imaging modes having different transmit power requirements, if the transmit power for one imaging mode is less than that provided by the power supply voltage, the pulse width modulation signal is generated to cause a reduction in the width of the pulses in each burst of the waveform generated by the transmit FETs 16A, 16B, . . . 16N for the duration of that imaging mode. The pulse width modulating of the waveforms for the imaging modes is effective to set emitted acoustic power and heating of the transmitting elements to proper values for the different imaging modes.

An advantage of the invention resulting from the fact that the transmit voltage for all of the different imaging modes is not reduced is that each imaging mode can be operable at full sensitivity on a line-by-line basis. This avoids the need to switch the transmit voltage on a line-by-line basis which is difficult if not impossible at high imaging speeds.

Nevertheless, the power supply voltage is normally varied based upon user controls, such as the location of the transmit focal point and the width or depth of the ultrasonic image. However, these settings do not occur on a line-by-line basis or even on a frame-by-frame basis, but in response, for example, to a user changing some system control. Therefore, the power supply voltage is not normally strictly fixed and the system can control this voltage over a reasonably wide range. Once the voltage has been established for the system control settings, it becomes effectively fixed on a line-by-line basis in which case, the invention is capable of maintaining this fixed power supply voltage while allowing imaging in multiple modes at optimum sensitivity.

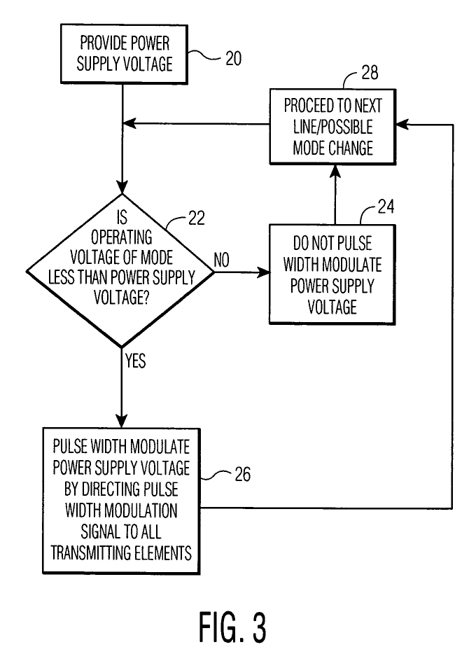

FIG. 3 shows a flow chart of the manner in which pulse width modulation of the transmit amplitude is applied in the invention. At step 20, the power supply voltage is provided, e.g., by the transmit power supply 12, and at step 22, a determination is made whether the transmit power of the imaging mode is less than that provided by the power supply voltage. If not, at step 24, a pulse width modulation signal which would cause a reduction in the width of the pulses of each burst of the waveform generated by the transmitting elements is not sent by the timing generator 14. On the other hand, if the transmit power of an imaging mode is less than that provided by the power supply voltage, then at step 26, a pulse width modulation signal would be generated by the timing generator 14, which would cause the transmit FETs 16A, 16B, . . . 16N to create a waveform with pulses having a reduced width, and sent to the transmit FETs 16A, 16B, . . . 16N. The process then continues by proceeding to the next line at step 28, which might be performed at a different imaging mode, and then repeating the determination of whether the transmit power is less than that provided by the power supply voltage. The process continues on the line-by-line basis until the imaging is completed.

In one preferred embodiment, the same circuitry used to pulse width modulate the transmit power, i.e., the timing generator 14, is also used for pulse width apodization.

Although illustrative embodiments of the present invention have been described herein with reference to the accompanying drawings, it is to be understood that the invention is not limited to these precise embodiments, and that various other changes and modifications may be effected therein by one of ordinary skill in the art without departing from the scope or spirit of the invention. For example, although as described above pulse width modulation is generally used to reduce the pulse width, it is conceivable that it could also be used to increase the pulse width, i.e., a system could be designed which uses very narrow pulse widths in a “normal” mode and then increases the pulse width for other mode.

1. A method for matching transmit voltages of different imaging modes of an ultrasonic transmitter having different transmit power requirements, comprising the steps of:
directing the same power supply voltage to all of the transmitting elements;
operatively switching between the imaging modes on a line-by-line basis; and
only when the requirement for the transmit power for an imaging mode is less than that provided by the power supply voltage, pulse width modulating the transmit waveform for that imaging mode by directing a pulse width modulation signal to all of the transmitting elements without changing the power supply voltage between different imaging modes.

2. The method of claim 1, wherein the transmitting elements are FETs.

3. The method of claim 1, further comprising the step of generating the pulse width modulating signals in a timing generator.

4. The method of claim 1, further comprising the step of generating the pulse width modulation signals based on analysis of the transmit power for the imaging modes relative to the power supply voltage.

5. The method of claim 1, wherein the switching between imaging modes occurs on a line-by-line basis.

6. The method of claim 1, further comprising the steps of:
providing circuitry for pulse width modulating the transit waveforms; and
using the same circuitry for pulse width apodization.

7. The method of claim 1, wherein the pulse width modulation signals directed to the transmitting elements are effective to set emitted acoustic power and heating of the transmitting elements to proper values for the imaging modes.

8. A medical ultrasound imaging system operable in multiple imaging modes on a line-by-line basis and which have different transmit power requirements, comprising:
a plurality of transmitting elements each generating a waveform;
a power supply coupled by power supply rails to said transmitting elements such that the same power supply voltage is directed to all of said transmitting elements for all of the different imaging modes;
a timing generator coupled to said transmitting elements and arranged to generate and direct pulse width modulation signals to said transmitting elements for imaging modes having a transmit power less than that provided by the power supply voltage

9. The imaging system of claim 8, wherein said timing generator is arranged to generate pulse width modulation signals which cause a reduction in the width of bursts in pulses of the waveforms generated by said transmitting elements.

10. The imaging system of claim 8, wherein said transmitting elements are FETs.

11. The imaging system of claim 8, wherein said timing generator is arranged to generate the pulse width modulation signals based on analysis of the transmit power for the imaging modes relative to the power supply voltage.

12. The imaging system of claim 8, further comprising a control computer for storing events for control changes and events for each line of an imaging procedure and providing event commands to said power supply and said timing generator.

13. The imaging system of claim 8, wherein said timing generator is used for pulse width apodization.

14. The imaging system of claim 8, wherein said timing generator is arranged to generate pulse width modulation signals effective to set emitted acoustic power and heating of said transmitting elements to proper values for the imaging modes.
